(19) United States
(12) Patent Application Publication
(10) Pub. No.: US 2026/0253212 A1
(43) Pub. Date: Aug. 27, 2026

(54) COMPUTER-AIDED DIAGNOSIS SYSTEM

(71) Applicant: GYRUS ACMI, INC. D/B/A OLYMPUS SURGICAL TECHNOLOGIES AMERICA, Westborough, MA (US)

(72) Inventors: Thorsten Juergens, Hamburg (DE); Frank Filiciotto, Bethlehem, PA (US)

(21) Appl. No.: 19/159,388

(22) PCT Filed: Feb. 8, 2024
(86) PCT No.: PCT/US2024/015003

§ 371 Date: Aug. 25, 2025

Related U.S. Application Data
(60) Provisional application No. 63/488,564, filed on Mar. 6, 2023.

(51) Int. Cl. G06T 7/00; A61B 1/00; A61B 1/05; G06T 7/70; G06T 11/10; G06T 11/23; G06V 20/52; G06V 40/10; G16H 15/00; G16H 50/20

(52) U.S. Cl. CPC G06T 7/0012; A61B 1/00045; A61B 1/05; G06T 11/10; G06T 11/23; G06V 20/52; G06V 40/10; G16H 15/00; G16H 50/20; G06T 7/70; G06T 2207/10016; G06T 2207/10068; G06T 2207/30201; G06T 2207/30232; G06T 2210/12; G06V 2201/03

(57) ABSTRACT

A system for intelligent surveillance of attention and recognition of computer-aided diagnosis system outputs. The system can include an endoscope, a computer-aided diagnostic module, a camera, a memory, and a controller. The endoscope can include an elongated member that can include a distal portion and a process camera attached to the distal portion. The process camera can capture a video stream during a procedure. The computer-aided diagnostic module can be configured to detect an abnormality within the video stream using a diagnostic algorithm and transmit a signal. The controller can be configured to determine a gaze location of the doctor during the procedure using a gaze algorithm, determine whether the doctor looked at the detected abnormality by comparing the signal from the computer-aided diagnostic module and the gaze location of the doctor, and trigger a countermeasure based on determining that the doctor did not look at the detected abnormality.

### PRIORITY CLAIM

This application claims the benefit of priority to U.S. Provisional Patent Application Ser. No. 63/488,564, filed Mar. 6, 2023, the contents of which are incorporated herein by reference in their entirety.

### TECHNICAL FIELD

This disclosure generally relates to endoscopic systems and, more particularly, to endoscopic systems for surveillance of attention and recognition of computer-aided diagnosis system outputs.

### BACKGROUND

Conventional endoscopes can be used in a variety of clinical procedures. For example, endoscopes can be used for illuminating, imaging, detecting and diagnosing one or more disease states, providing fluid delivery (e.g., saline or other preparations via a fluid channel) toward an anatomical region, providing passage (e.g., via a working channel) of one or more therapeutic devices for sampling or treating an anatomical region, providing suction passageways for collecting fluids (e.g., saline or other preparations), and the like. Such anatomical regions can include the gastrointestinal tract (e.g., esophagus, stomach, duodenum, pancreaticobiliary duct, intestines, colon, and the like), renal area (e.g., kidney(s), ureter, bladder, urethra), other internal organs (e.g., reproductive systems, sinus cavities, submucosal regions, respiratory tract), and the like.

### BRIEF DESCRIPTION OF THE DRAWINGS

Various examples are illustrated in the figures of the accompanying drawings. Such examples are demonstrative and not intended to be exhaustive or exclusive examples of the present subject matter.

FIG. 1 illustrates a schematic diagram of an endoscopy system, according to an example of the present disclosure.

FIG. 2 illustrates a schematic diagram of the imaging and control system of FIG. 1, showing the imaging and control system connected to the endoscope, according to an example of the present disclosure.

FIG. 3 is a block diagram illustrating a system, according to an example of the present disclosure.

FIG. 4 is a schematic diagram illustrating a system, according to an example of the present disclosure.

FIG. 5 is a flowchart illustrating a method, according to an example of the present disclosure.

FIG. 6 is a flowchart further illustrating the method from FIG. 5, according to an example of the present disclosure.

FIG. 7 is a schematic diagram illustrating an example display based on a gaze location of a medical professional, according to an example of the present disclosure.

FIG. 8 is a schematic diagram illustrating an example display based on a gaze location of a medical professional, according to an example of the present disclosure.

FIG. 9 is a flowchart further illustrating the method from FIG. 5, according to an example of the present disclosure.

FIG. 10 is a schematic diagram illustrating an example display based on a gaze location of a medical professional, according to an example of the present disclosure.

FIG. 11 is a schematic diagram illustrating an example display based on a gaze location of a medical professional, according to an example of the present disclosure.

FIG. 12 illustrates a schematic diagram of an example of an annotated image that can be displayed on a display within an operating room, according to an example of the present disclosure.

FIG. 13 is a flowchart further illustrating the method from FIG. 5, according to an example of the present disclosure.

FIG. 14 is a block diagram illustrating an example of a machine upon which one or more examples can be implemented.

### DETAILED DESCRIPTION

Medical examinations can include using various medical imaging techniques to capture images of a particular part of a patient's body for visual examination by a physician. For example, a colonoscopy can involve the endoscopic examination of the large bowel of the patient with a camera positioned on a distal tip of a steerable probe. The steerable probe can include a working channel through which instruments can be passed to access the area under inspection. Such medical examinations can facilitate the physician providing a visual diagnosis of anomalous tissues (e.g., polyps) and the opportunity for biopsy or removal of suspected cancerous tissues.

Computer-aided detection (CADe) or computer-aided diagnosis (CADx) systems (collectively referred to as “CAD systems”) can be deployed in real-time during a medical examination to increase both the efficiency and efficacy of the medical examination. CAD systems can assist physicians in interpreting medical images by processing the medical images and generating annotations that highlight anomalous tissues (e.g., in the form of virtual bounding boxes). The inventors of the present disclosure have discovered at least the following problems with current systems including CAD systems. Even with the presentation of such annotations, there is a risk that a physician will not carefully examine, or even notice every anomalous tissue. Furthermore, even when a physician does examine an individual anomalous tissue and determine whether an action is warranted (e.g., polyp removal, biopsy, or the inclusion of diagnosis in an examination report, etc.), there may be inefficiencies associated with documenting these determined actions in association with the individually identifies anomalous tissues.

Generally speaking, the present disclosure includes a system that can generate signals for an operator of a CAD system based on an amount of time the operator spends examining individual CAD-identified tissue anomalies. In an example, a CAD system can detect when an operator may have overlooked a tissue anomaly (e.g., a colon polyp) displayed on a monitor during a medical examination (e.g., a colonoscopy). Then, when an operator appears to have overlooked a tissue anomaly, the system can generate a user-perceptible signal that can be designed to draw the operator's attention to the tissue anomaly.

For example, a system for intelligent surveillance of attention and recognition of computer-aided diagnosis system outputs can include an endoscope, a computer-aided diagnostic module, a camera, a memory, and a controller. The endoscope can include an elongated member can include a distal portion and a process camera attached to the distal portion. The process camera can capture a video stream during a procedure. The computer-aided diagnostic module can be configured to detect an abnormality within the video stream using a diagnostic algorithm and transmit a signal. The camera can capture an operator video stream. The operator video stream can include at least eyes of a doctor during the procedure.

The memory can include instructions. The controller can include processing circuitry that, when in operation, is configured by the instructions to determine a gaze location of the doctor during the procedure using a gaze algorithm, determine whether the doctor looked at the detected abnormality by comparing the signal from the computer-aided diagnostic module and the gaze location of the doctor, and trigger a countermeasure based on determining that the doctor did not look at the detected abnormality. The gaze location can indicate a location on a monitor that the doctor looks at during the procedure.

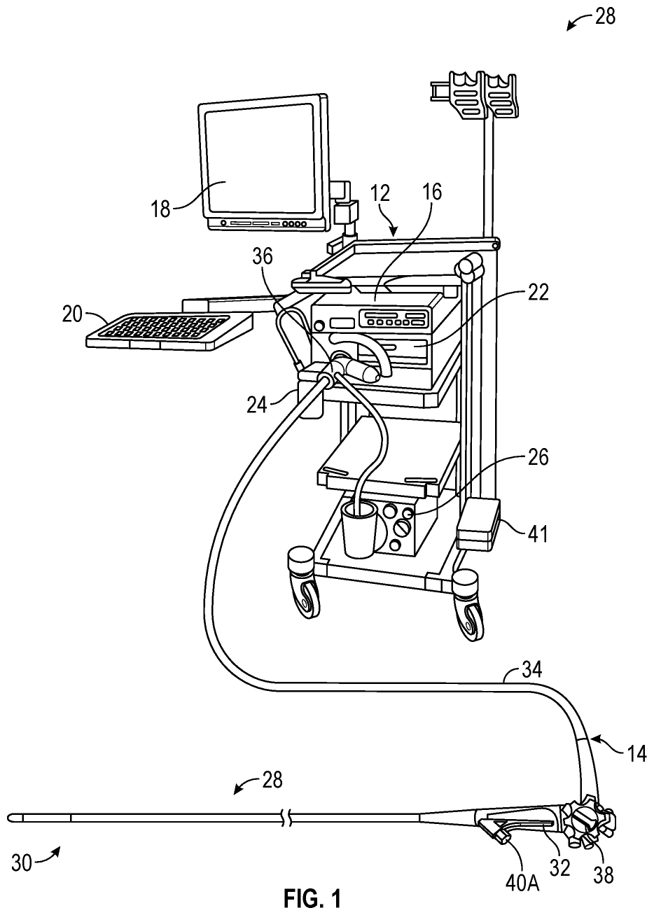

FIG. 1 is a schematic diagram of an endoscopy system 10 that can include an imaging and control system 12 and an endoscope 14. The system 10 is an illustrative example of an endoscopy system suitable for use with the systems, devices, and methods described herein, such as a colonoscope system for automatically annotating endoscopic videos.

The endoscope 14 can be insertable into an anatomical region for imaging or to provide passage of or attachment to (e.g., via tethering) one or more sampling devices for biopsies or therapeutic devices for treatment of a disease state associated with the anatomical region. The endoscope 14 can interface with and connect to imaging and control system 12. The endoscope 14 can also include a colonoscope, though other types of endoscopes can be used with the features and teachings of the present disclosure. The imaging and control system 12 can include a control unit 16, an output unit 18, an input unit 20, a light source unit 22, a fluid source 24, and a suction pump 26.

The imaging and control system 12 can include various ports for coupling with the endoscopy system 10. For example, the control unit 16 can include a data input/output port for receiving data from and communicating data to the endoscope 14. The light source unit 22 can include an output port for transmitting light to the endoscope 14, such as via a fiber optic link. The fluid source 24 can include a port for transmitting fluid to the endoscope 14. The fluid source 24 can include, for example, a pump and a tank of fluid or can be connected to an external tank, vessel, or storage unit. The suction pump 26 can include a port to draw a vacuum from the endoscope 14 to generate suction, such as for withdrawing fluid from the anatomical region into which the endoscope 14 is inserted. The output unit 18 and the input unit 20 can be used by an operator of the endoscopy system 10 to control functions of the endoscopy system 10 and view the output of the endoscope 14. The control unit 16 can also generate signals or other outputs from treating the anatomical region into which the endoscope 14 is inserted. In some examples, the control unit 16 can generate electrical output, acoustic output, fluid output, and the like for treating the anatomical region with, for example, cauterizing, cutting, freezing, and the like.

The endoscope 14 can include an insertion section 28, a functional section 30, and a handle section 32, which can be coupled to a cable section 34 and a coupler section 36. The insertion section 28 can extend distally from the handle section 32, and the cable section 34 can extend proximally from the handle section 32. The insertion section 28 can be elongated and can include a bending section and a distal end to which the functional section 30 can be attached. The bending section can be controllable (e.g., by a control knob 38 on the handle section 32) to maneuver the distal end through tortuous anatomical passageways (e.g., stomach, duodenum, kidney, ureter, etc.). The insertion section 28 can also include one or more working channels (e.g., an internal lumen) that can be elongated and can support the insertion of one or more therapeutic tools of the functional section 30, such as a cholangioscope. The working channel can extend between the handle section 32 and the functional section 30. Additional functionalities, such as fluid passages, guide wires, and pull wires, can also be provided by the insertion section 28 (e.g., via suction or irrigation passageways or the like).

A coupler section 36 can be connected to the control unit 16 to connect to the endoscope 14 to multiple features of the control unit 16, such as the input unit 20, the light source unit 22, the fluid source 24, and the suction pump 26.

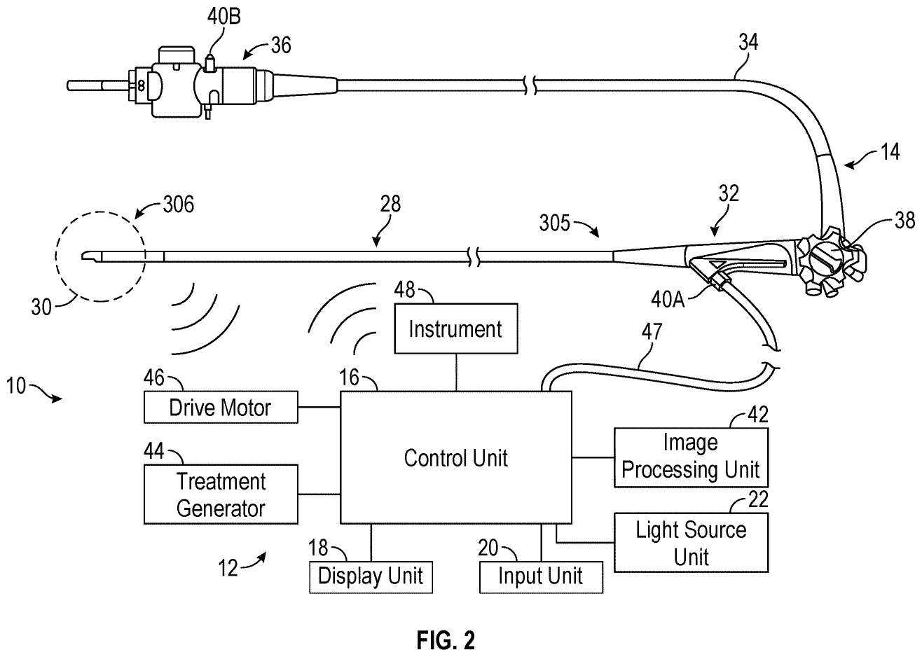

The handle section 32 can include the knob 38 and the port 40A. The knob 38 can be connected to a pull wire or other actuation mechanisms that can extend through the insertion section 28. The port 40A, as well as other ports, such as a port 40B (FIG. 2), can be configured to couple various electrical cables, guide wires, auxiliary scopes, tissue collection devices, fluid tubes, and the like to the handle section 32, such as for coupling with the insertion section 28.

According to examples, the imaging and control system 12 can be provided on a mobile platform (e.g., a cart 41) with shelves for housing the light source unit 22, the suction pump 26, an image processing unit 42 (FIG. 2), etc. Alternatively, several components of the imaging and the control system 12 (shown in FIGS. 1 and 2) can be provided directly on the endoscope 14 to make the endoscope “self-contained.”

The functional section 30 can include components for treating and diagnosing anatomy of a patient. The functional section 30 can include an imaging device, an illumination device, and an elevator. The functional section 30 can further include optically enhanced biological matter and tissue collection and retrieval devices as described herein. For example, the functional section 30 can include one or more electrodes conductively connected to the handle section 32 and functionally connected to the imaging and control system 12 to analyze biological matter in contact with the electrodes based on comparative biological data stored in the imaging and control system 12.

FIG. 2 is a schematic diagram of the endoscopy system 10 of FIG. 1 including the imaging and control system 12 and the endoscope 14. FIG. 2 schematically illustrates components of the imaging and the control system 12 coupled to the endoscope 14, which in the illustrated example includes a colonoscope. The imaging and control system 12 can include the control unit 16, which can include or be coupled to an image processing unit 42, a treatment generator 44, and a drive unit 46, as well as the light source unit 22, the input unit 20, and the output unit 18. The control unit 16 can include, or can be in communication with, an endoscope, a surgical instrument 48, and an endoscopy system, which can include a device configured to engage tissue and collect and store a portion of that tissue and through which imaging equipment (e.g., a camera) can view target tissue via inclusion of optically enhanced materials and components. The control unit 16 can be configured to activate a camera to view target tissue distal of the endoscopy system. Likewise, the control unit 16 can be configured to activate the light source unit 22 to shine light on the surgical instrument 48, which can include select components configured to reflect light in a particular manner, such as enhanced tissue cutters with reflective particles.

The coupler section 36 can be connected to the control unit 16 to connect to the endoscope 14 to multiple features of the control unit 16, such as the image processing unit 42 and the treatment generator 44. In examples, the port 40A can be used to insert another surgical instrument 48 or device, such as a daughter scope or auxiliary scope, into the endoscope 14. Such instruments and devices can be independently connected to the control unit 16 via the cable 47. In examples, the port 40B can be used to connect coupler section 36 to various inputs and outputs, such as video, air, light, and electric.

The image processing unit 42 and light source unit 22 can each interface with the endoscope 14 (e.g., at the functional section 30) by wired or wireless electrical connections. The imaging and control system 12 can accordingly illuminate an anatomical region, collect signals representing the anatomical region, process signals representing the anatomical region, and display images representing the anatomical region on the display unit 18. The imaging and control system 12 can include the light source unit 22 to illuminate the anatomical region using light of desired spectrum (e.g., broadband white light, narrow-band imaging using preferred electromagnetic wavelengths, and the like). The imaging and control system 12 can connect (e.g., via an endoscope connector) to the endoscope 14 for signal transmission (e.g., light output from light source, video signals from imaging system in the distal end, diagnostic and sensor signals from a diagnostic device, and the like).

The fluid source 24 (shown in FIG. 1) can be in communication with control unit 16 and can include one or more sources of air, saline, or other fluids, as well as associated fluid pathways (e.g., air channels, irrigation channels, suction channels, or the like) and connectors (barb fittings, fluid seals, valves, or the like). The fluid source 24 can be utilized as an activation energy for a biasing device or a pressure-applying device of the present disclosure. The imaging and control system 12 can also include the drive unit 46, which can include a motorized drive for advancing a distal section of endoscope 14.

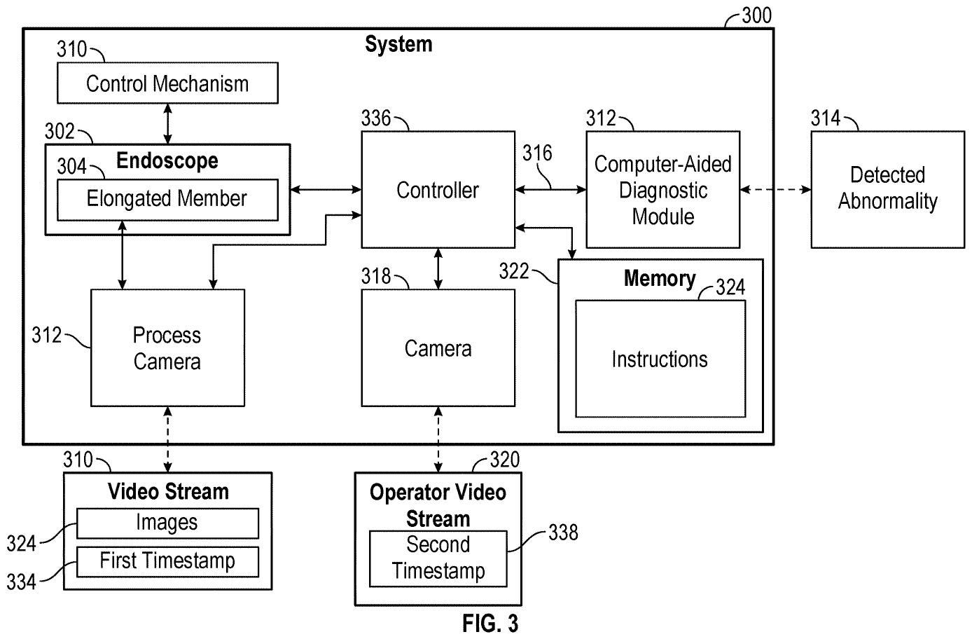

FIG. 3 is a block diagram that describes a system 300, according to an example of the present disclosure. The system 300 can include an endoscope 302, a computer-aided diagnostic module 312, a camera 318, a memory 322, and a controller 336. The elongated member 304 can include a distal portion 306 and a process camera 308 attached to the distal portion 306. The memory 322 can include instructions 324. As best shown in FIG. 2, the elongated member 304 (e.g., the insertion section 28 and the functional section 30 (FIGS. 1 and 2)) can extend from a proximal portion 305 to a distal portion 306. The elongated member 304 can be insertable into a cavity of a patient.

The process camera 308 can be configured to capture a process video stream 310 during a medical procedure. The image processing unit 42 (FIG. 2) can process the video stream 310 and display the video stream 310 on the display unit 18 (FIGS. 1 and 2) so doctors, or other medical professionals, can see in front of the distal portion 306 of the elongated member 304 during the medical procedure. The camera 308 can also simultaneously transmit the video stream 310 to multiple components. For example, the camera 308 can transmit the video stream 310 to the display unit to provide a live feed of the video stream 310 on the display for the doctor, the image processing unit or the controller 336 for processing, and the memory 322 for storage of a raw version of the video stream 310. Any example of the video stream 310 can include a first timestamp 334 to help sync the video stream 310 with other signals of the system 300.

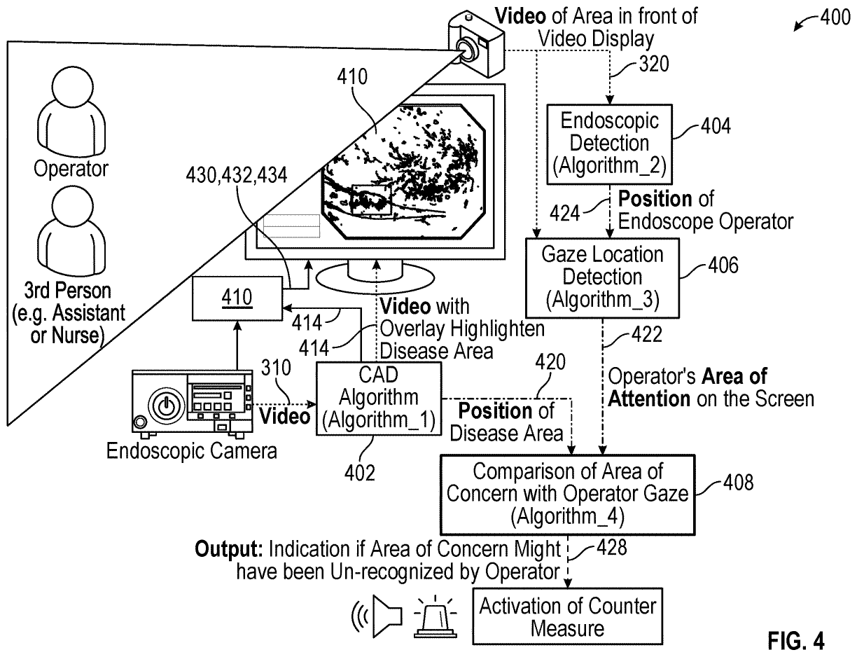

The computer-aided diagnostic module 312, which will be discussed in more detail with reference to FIG. 4 below, can be configured to detect an anomoly or an abnormality (detected abnormality 314) within the process video stream 310 using a diagnostic algorithm and transmit a signal 316. In examples, the computer-aided diagnostic module 312 can be a module in communication with the controller 336, which can run the detected abnormality 314 to diagnose the video stream 310 during the medical procedure automatically. In another example, the computer-aided diagnostic module 312 can be a separate controller of the system 300. As previously discussed, the computer-aided diagnostic module 312 can be deployed in real-time during a medical examination to increase the efficiency and efficacy of the medical examination. The computer-aided diagnostic module 312 can assist a doctor in interpreting medical images by processing the medical images and generating annotations that highlight anomalous tissues (e.g., in the form of virtual bounding boxes).

The camera 318 can be one or more cameras mounted around the system 300 to capture at least the doctor performing the medical procedure. For example, the camera 318 can be configured to capture the eyes of the doctor performing the medical procedure. The camera 318 can capture an operator video stream 320. The operator video stream 320 can be a video stream of the doctor completing the medical procedure. The operator video stream 320 can be used for training purposes and for processing purposes. As such, the camera 318 can transmit the operator video stream 320 to multiple components of the system 300. For example, the camera 318 can transmit the operator video stream 320 to the display unit to show a live stream of the operator video stream 320. The camera 318 can also transmit the operator video stream 320 to a video processor so that the video processor can analyze the operator video stream 320. In yet another example, the camera 318 can send the operator video stream 320 to the controller 336, or to any other component of the system 300. The operator video stream 320 can include a second timestamp 338 to help sync the operator video stream 320 with the video stream 310 or any other signal of the system 300.

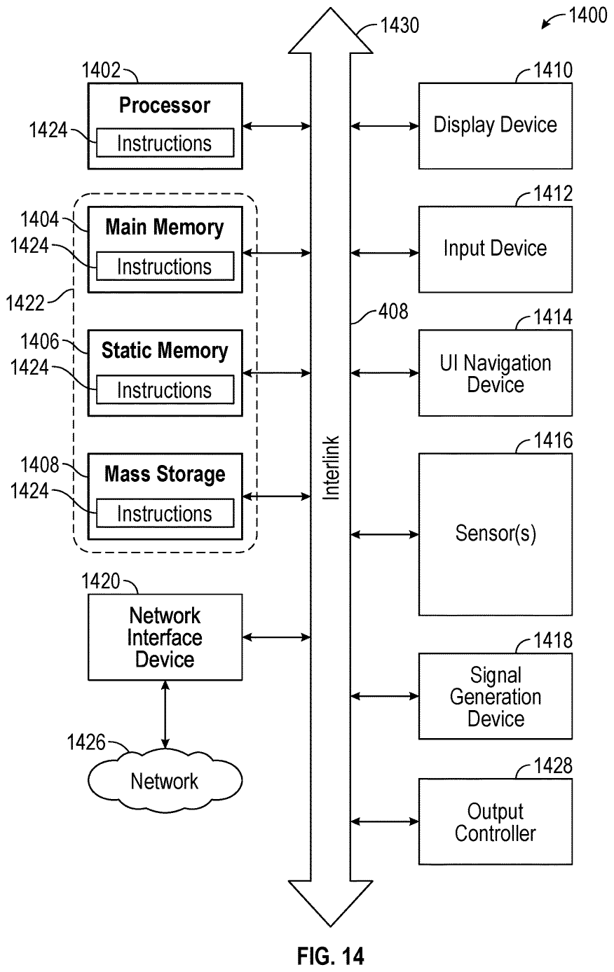

The memory 322 can be a main memory, a static memory, or a mass storage device (e.g., a main memory 1404, static memory 1406, or a mass storage 1408 as discussed with reference to FIG. 14). The memory 322 can include the instructions 324, which can include a program, process, or other action that can configure the controller 336 to complete tasks to help the system 300 complete the surveillance of attention and recognition of computer-aided diagnosis system outputs.

The controller 336 (e.g., the control unit 16) can be one or more controllers configured to operate the system 300. The instructions 324 on the memory 322, can cause the processing circuitry of the controller 336 to complete operations or procedures. For example, the processing circuitry of the controller 336 can be configured by the instructions 324 to determine a gaze location of the doctor during the endoscopic procedure using a gaze algorithm, determine whether the doctor looked at the detected abnormality by comparing the signal from the computer-aided diagnostic module and the gaze location of the doctor; and trigger a countermeasure based on determining that the doctor did not look at the detected abnormality.

For example, the determination that the operator may have overlooked the tissue anomaly can include analyzing medical images using with a CAD system to identify the tissue anomaly, assigning an anomaly ID to the tissue anomaly (e.g., a unique identifier that specifically corresponds to the individual tissue anomaly), monitoring an eye gaze of the operator to determine a gaze duration associated with the anomaly ID (e.g., an amount of time that the operator focused on the tissue anomaly on a monitor), and comparing the gaze duration to one or more gaze duration thresholds. In some examples, the gaze duration being below a gaze duration threshold can indicate that the operator has overlooked the tissue anomaly. The system 300 will be discussed in more detail herein with reference to FIGS. 4-14.

FIG. 4 is a schematic diagram illustrating a system, according to an example of the present disclosure. The system 400 can be an example of an implementation of the system 300 from FIG. 3. The system 300 and the system 400 can be discussed in combination herein as the systems. In addition to the components of the system 300, the system 400 can include a user interface module 410 and a display 412. The system 400 can also include a CAD algorithm 402, an endoscopist detection algorithm 404, a gaze location detection algorithm 406, and a comparison algorithm 408 stored as instructions 324 (FIG. 3) on the memory 322 (FIG. 3) that can be executed by the controller 336 to complete tasks.

The CAD algorithm 402 can be initiated by the computer-aided diagnostic module 312, the controller 336, or any other controller or processor of, or in communication with, the systems. The CAD algorithm 402 can be configured to use the video stream 310 (FIG. 3) from the process camera 308 (FIG. 3) and analyze the video stream 310 to find, for example, the detected abnormality 314 (FIG. 3), which can be a polyp, anomalies, diseases, other non-desirable features, or the like, within the patient.

In examples, the CAD algorithm 402 can determine a location of the detected abnormality 314 and can label the location of the detected abnormality 314. For example, the CAD algorithm 402 can save a position of the abnormality 420 on the video stream 310, which can be saved on the memory 322 (FIG. 3), transmitted to any other component of the systems, or shared, saved, or transmitted to a device on the cloud. Moreover, the CAD algorithm 402 can label the detected abnormality 314 with a unique identifier that can help identify the detected abnormality 314 on future procedures, and during pathological testing of the detected abnormality 314. The identification of the detected abnormality 314 can also be stored with the position of the abnormality 420 such that any system that receives the position of the abnormality 420 also has the unique identifier of the detected abnormality 314.

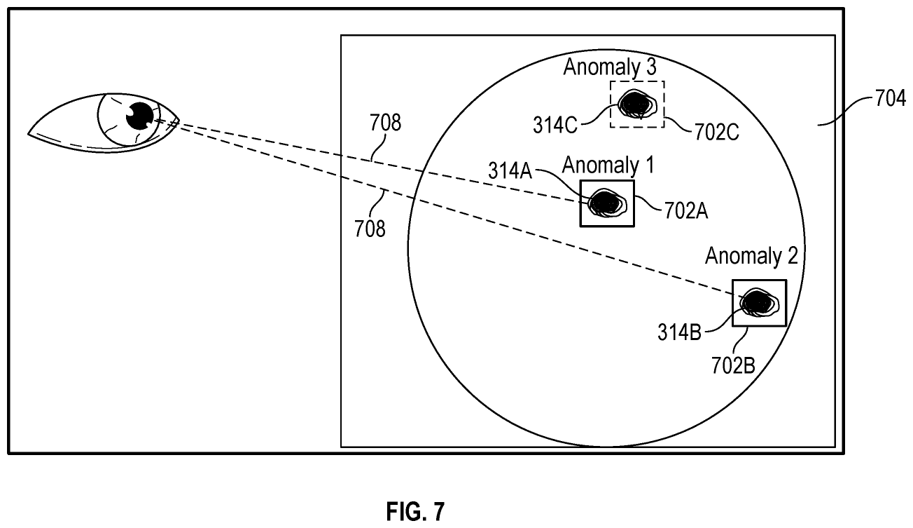

In examples, the CAD algorithm 402 can put a bounding box (discussed further in FIG. 7) around the detected abnormality 314 to create a visual indicia of a detected abnormality 314 on an analyzed video stream 414. In examples, the bounding box can also include the unique identification of the detected abnormality 314 such that the unique identification can be displayed or indicated around the bounding box. The CAD algorithm 402 can direct the computer-aided diagnostic module 312, the controller 336, or other processor, to transmit the analyzed video stream 414 to the user interface module 410 for further processing, which will be discussed below.

The endoscopist detection algorithm 404 can be initiated by the controller 336 or any other processor of, or in communication with, the system 300, the system 400. The endoscopist detection algorithm 404 can configure a processor to receive the operator video stream 320 (FIG. 3) from the camera 318 (FIG. 3) to analyze the operator video stream 320 and determine a position of an endoscope operator 424. The position of the endoscope operator 424 can be the person that is holding the endoscope. The endoscopist detection algorithm 404 can be trained via machine learning techniques to find the position of the endoscope operator 424 by learning the physical appearance of all doctors that use the systems. In another example, the endoscopist detection algorithm 404 can find the endoscope and determine the person that is holding the endoscope.

In examples, once the endoscopist detection algorithm 404 determines the operator of the endoscope, the endoscopist detection algorithm 404 can direct the processor to focus the camera 318 on the endoscopist. Specifically, the endoscopist detection algorithm 404 can direct the processor to focus the camera 318 on the face of the endoscopist to ensure the eyes of the operator are within the frame of the operator video stream 320.

Accuracy of the endoscopist detection algorithm 404 can help prevent false alarms in the systems. For example, the endoscopist detection algorithm 404 can ensure that the systems are looking at the eyes of the medical professional performing the medical procedure, not a nurse, physician assistant, or any other person in the procedure room during the medical procedure.

In examples, after the endoscopist detection algorithm 404 finds the location of the endoscope operator 424, the endoscopist detection algorithm 404 can determine if the eyes of the endoscope operator are within the operator video stream 320. If the eyes of the operator are not within the operator video stream 320, endoscopist detection algorithm 404 can generate an alarm, signal, or warning to ask for the endoscopist to move into a new position, for the camera 318 to be moved, or for an object that is obstructing the view of the camera 318 to be moved.

The gaze location detection algorithm 406 can be ran by the controller 336 or any other processor of, or in communication with, the system 300, the system 400. In examples, the gaze location detection algorithm 406 can be run once the endoscopist detection algorithm 404 finds the location of the endoscope operator 424. The gaze location detection algorithm 406 can configure a processor to receive the operator video stream 320 (FIG. 3) from the camera 318 (FIG. 3) to analyze the operator video stream 320 and determine a gaze location 426 (FIG. 3) of the medical professional performing the medical procedure. For example, the endoscopist detection algorithm 404 can be used to find an area of attention 422, which can be a location on the display 412 that the eyes of the medical professional performing the medical procedure gaze during the medical procedure.

In another example, the operator of the endoscope can wear glasses, a headset, or any other sensor that can help the systems determine a direction of gaze of the endoscopist. For example, the endoscopist can wear glasses that can include the camera 318 mounted thereon. As such, the operator video stream 320 can then be stored and used for further machine learning of the CAD algorithm 402 or any other algorithm or system that can benefit from seeing where the doctor is gazing during the medical procedure. Moreover, when the doctor performing the medical procedure is wearing the glasses, the endoscopist detection algorithm 404 can be used to find the position of the doctor performing the medical procedure with relation to the display 412. For example, the glasses can include sensors that can detect position, orientation, distances, or acceleration, to help determine the position of the doctor performing the medical procedure in relation to the display 412.

The glasses can include AR or VR technology such that any of the video streams of the systems can be displayed onto the lens of the glasses. Moreover, the glasses can communicate warnings, signals, or other visual indicators to the doctor performing the medical procedure. As such, the glasses can have a controller in communication with any of the controllers or processors of the systems.

The comparison algorithm 408 can be run by the computer-aided diagnostic module 312, the controller 336, or any other controller or processor of, or in communication with, the systems. The comparison algorithm 408 can be configured to compare one or more signals, characteristics, or other parameters generated, captured, or detected by components of the systems. For example, the comparison algorithm 408 can configure processors of the systems to compare the position of abnormality 420 and the area of attention 422. For example, the position of abnormality 420 and the area of attention 422 can be compared as the position of abnormality 420 and the area of attention 422 are displayed on the display 412.

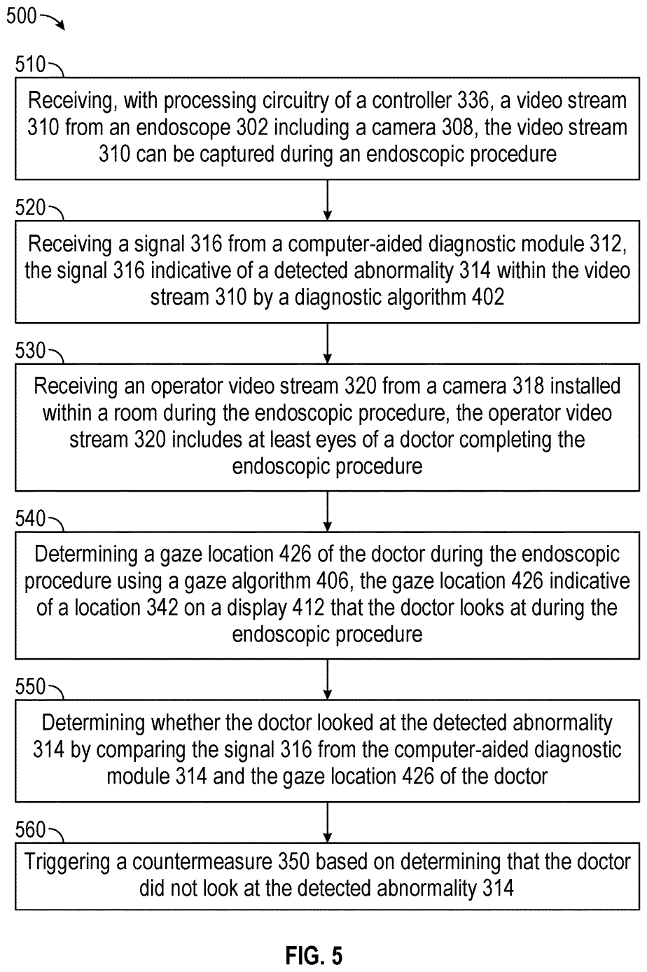

In an example, if the position of abnormality 420 and the area of attention 422 do not overlap for a minimum amount of time (e.g., for a threshold time) during the time that the detected abnormality 314 is displayed on the display 412, the comparison algorithm 408 can generate a signal, warning, or other indication that the detected abnormality 314 may have been missed by the medical professional performing the medical procedure. In examples, the comparison algorithm 408 can generate audible warning signals, visible warning signals, a still image in which the unrecognized disease area is displayed, or the like, to generate a warning video stream 428. The comparison algorithm 408 will be discussed in more detail with reference to FIGS. 5-11.

The user interface module 410 can receive the video stream 310, the operator video stream 320, the analyzed video stream 414, and the warning video stream 428 from the comparison algorithm 408. In examples, the user interface module 410 can combine, alter, or edit the video stream 310, the analyzed video stream 414, or the warning video stream 428. For example, the user interface module 410 can overlay the warning video stream 428 onto the video stream 310 to generate an overlaid video stream 430. The user interface module 410 can overlay the video stream 310 with the analyzed video stream 414 to generate a detected video stream 432. Moreover, the user interface module 410 can overlay the detected video stream 432 with the warning video stream 428 to generate a detected and warning video stream 434.

The detected and warning video stream 434 can include both the detected abnormality 314 from the CAD algorithm 402 and the warning video stream 428, which can include warnings generated by the comparison algorithm 408. In such an example, the user interface module 410 can continuously update the detected and warning video stream 434 as the user interface module 410 receives the video stream 310, the analyzed video stream 414, or the detected and warning video stream 434.

The user interface module 410 can transmit any combination of the video stream 310, the operator video stream 320, the analyzed video stream 414, or the detected and warning video stream 434 to the display 412, the operator video stream 320, or any other components of, or in communication with, the systems. For example, the user interface module 410 can transmit the video stream 310, the operator video stream 320, the analyzed video stream 414, or the detected and warning video stream 434 to the memory 322 for storage.

The display 412 (e.g., the output unit 18 (FIG. 1) or the display unit 18 (FIG. 2)), can be located within the room that the medical procedure is being performed. The display can be in communication with the user interface module 410 or any other component of the systems to communicate with people within the room during the medical procedure.

FIG. 5 is a flowchart illustrating a method 500, according to an example of the present disclosure. The method 500 enables the systems (e.g., the system 300 from FIG. 3 or the system 400 from FIG. 4) to complete the surveillance of attention and recognition of computer-aided diagnosis system outputs. The method 500 includes an operation to trigger a countermeasure when the system detects an abnormality that was not inspected by the endoscopist. In examples, the method 500 can include any of steps 510-560.

At step 510, the method 500 can include receiving, with processing circuitry of the controller, the video stream from the process camera installed on the endoscope, the video stream captured during an endoscopic procedure. For example, the processing circuitry of the controller 336 can receive the video stream 310 from the process camera 308. As discussed herein, the process camera 308 that can be installed on the endoscope 302. The process camera 308 can be installed on the elongated member 304 of the endoscope 302. The video stream 310 can be captured during an endoscopic procedure. The controller 336 can transmit the video stream 310 to multiple components of the system 300 simultaneously. For example, the controller 336 can send the video stream 310 to one or more of the memory 322 for storage, the computer-aided diagnostic module 312 for processing, a display (e.g., the display 412 of FIG. 4) within the room, or to any other component of the systems for further storage or processing.

At step 520, the method 500 can include receiving a signal from a computer-aided diagnostic module, the signal indicative of a detected abnormality within the video stream by a diagnostic algorithm. For example, the method 500 can include the controller 336 receiving the signal 316 from the computer-aided diagnostic module 312 or any other component of, or in communication with, the systems. The signal 316 can include one or more of the detected abnormality 314 that were detected by the computer-aided diagnostic module 312 or the controller 336 running the CAD algorithm 402. In examples, the computer-aided diagnostic module 312, the controller 336, or any other processor of, or in communication with, the systems can run the diagnostic algorithm 402, which can be stored directly on the computer-aided diagnostic module 312, on the memory 322, or on any other component of, or in communication with, the systems.

At step 530, the method 500 can include receiving an operator video stream from a camera installed within a room during the endoscopic procedure, the operator video stream includes at least eyes of a doctor completing the endoscopic procedure. For example, the method 500 can include the controller 336 receiving an operator video stream 320 from the camera 318 installed within a room during the endoscopic procedure. The operator video stream 320 can include at least eyes of the doctor completing the endoscoping procedure. The controller 336 can simultaneously transmit the operator video stream 320 to various components of the system 300. For example, the controller 336 can transmit the operator video stream 320 to the memory 322 for storage, the user interface module 410 (FIG. 4), or to any other component of the systems for processing.

At step 540, the method 500 can include determining a gaze location of the doctor during the endoscopic procedure using a gaze algorithm, the gaze location can be indicative of a location on a monitor that the doctor looks at during the endoscopic procedure. For example, the controller 336, when in operation, can be configured by the instructions 324 to determine a gaze location 426 of the doctor during the procedure using the gaze algorithm (e.g., the gaze location detection algorithm 406 of FIG. 4). The gaze location 426 can be indicative of a location 342 on a display 412 (e.g., the display unit 18 or any other monitor or display that the doctor looks at during the procedure).

At step 550, the method 500 can include determining whether the doctor looked at the detected abnormality by comparing the signal from the computer-aided diagnostic module and the gaze location of the doctor. For example, the controller 336 can determine whether the doctor looked at the detected abnormality 314 by comparing the signal 316 from the computer-aided diagnostic module 312 and the gaze location 426 of the doctor. For example, the comparison algorithm 408 (FIG. 4) can be run by the controller 336, the computer-aided diagnostic module 312, or any other processor of, or in communication with, the systems.

At step 560, the method 500 can include triggering a countermeasure based on determining that the doctor did not look at the detected abnormality. For example, the controller 336 can trigger a countermeasure 350 based on determining that the doctor did not look at the detected abnormality 314 over a threshold time. Triggering the countermeasure 350 can include transmitting a warning signal 352. The warning signal can indicate that the gaze location 426 of the doctor was not directed at the detected abnormality for a first set threshold time. In an example, the warning signal can include an abnormality identification unique to each detected abnormality by the computer-aided diagnostic module.

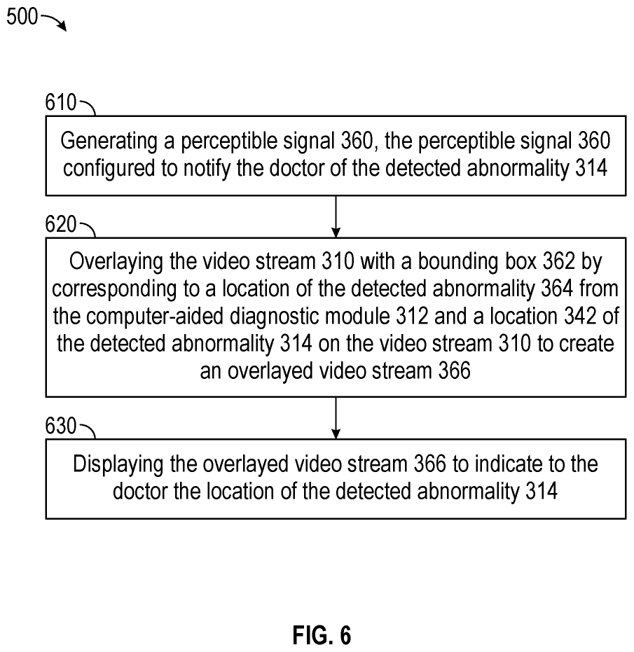

FIG. 6 is a flowchart further illustrating the method from FIG. 5, according to an example of the present disclosure. In an example, in triggering the countermeasure 350 of step 560, the method 500 can optionally include steps 610-630.

At step 610, step 560 of the method 500 can optionally include generating a perceptible signal 360. The perceptible signal 360 can be configured to notify the doctor of the detected abnormality (e.g., the detected abnormality 314). The perceptible signal 360, as discussed above, can be an audible signal, haptic signal, visual signal, any other kind of signal to get the attention of the doctor performing the medical procedure, or any combination thereof.

At step 620, step 560 of the method 500 can optionally include overlaying the video stream with a bounding box by corresponding a location of the detected abnormality from the computer-aided diagnostic module and a location of the detected abnormality on the video stream to create an overlayed video stream. For example, the controller 336 or an image processor (e.g., the image processing unit 42 or the user interface module 410 (FIG. 4)), can overlay the video stream 310 with a bounding box 362 by corresponding a location of the detected abnormality 314 from the computer-aided diagnostic module 312 and a location 342 of the detected abnormality 314 on the video stream 310 to create an overlayed video stream 366.

At step 630, the method 500 can include displaying the overlayed video stream 366 to indicate to the doctor, or other medical professionals, the location 342 of the detected abnormality 314.

In an example, the perceptible signal 360, or a user perceptible signal 360, can include: (i) adjusting bounding box parameters associated with the tissue anomaly to cause a bounding box to appear or become more conspicuous, (ii) generating an audible or tactile feedback signal, (ii) displaying a still image of the tissue anomaly which the operator appears to have overlooked, or the like. The system can draw the attention of the medical professional performing the procedure to individual tissue anomalies which have not received a sufficient gaze duration.

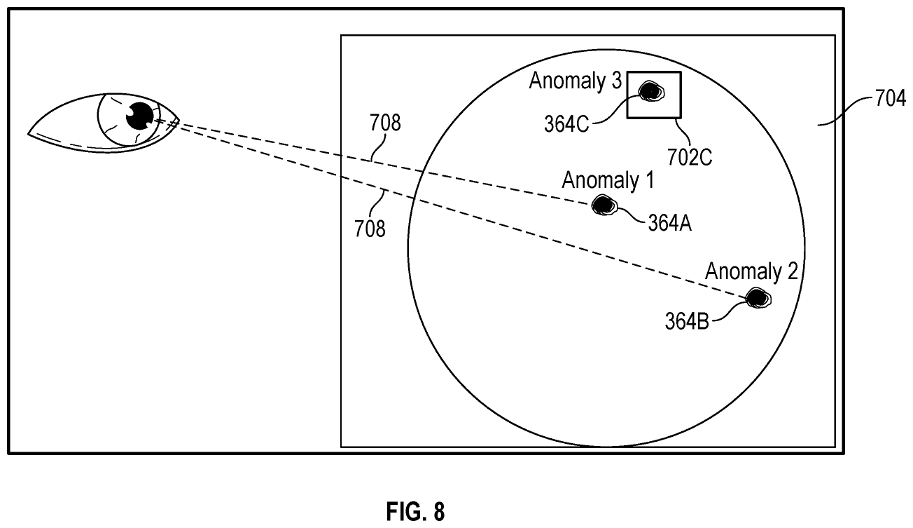

FIG. 7 is a schematic diagram illustrating an example display based on a gaze location of a medical professional, according to an example of the present disclosure. FIG. 8 is a schematic diagram illustrating an example display based on a gaze location of a medical professional, according to an example of the present disclosure. FIGS. 7 and 8 will be discussed together below.

In an example implementation, a bounding box 702 may be selectively modulated (e.g., color changes, flashing, etc.) for individual tissue anomalies. For example, the bounding box 702 can begin a first color, and if the medical professional completing the medical procedure does not look at the detected abnormality within the bounding box 702 over a threshold time, the bounding box 702 can change to a second color, begin to flash on the screen, grow in size, any other manipulation of the bounding box 702 that can help attract the attention of the medical professional completing the medical procedure, or the like. Such an implementation can prevent medical professionals from accidentally failing to examine CAD identified or classified tissue anomalies.

As shown in FIG. 7, a CAD system can identify or diagnose three tissue abnormalities 314A, 314B, and 314C, in a medical image 704. As discussed above, the bounding box 702 can be initially displayed in a first color, for example green. For example, the bounding box 702A can surround the tissue anomalies 314A, the bounding box 702B can surround the abnormality 314B, and the bounding box 702C can surround the tissue abnormality 314C.

For purposes of the example illustrated in FIG. 7, presume that the gaze duration of the medical professional completing the medical procedure on each of abnormality 314A and abnormality 314B exceeds a gaze duration threshold 708, and a gaze duration of the medical professional completing the medical procedure does not gaze upon abnormality 314C for at least the gaze duration threshold. Here, the bounding box 702A and the bounding box 702B surrounding the tissue abnormality 314A and the abnormality 314B, respectively, can be a first color, for example, green, and the bounding box 702C surrounding the tissue abnormality 314C can be a second color, for example, red. In another example, patterns, shapes, other visual indicia, or the like, can be used to indicate the gaze duration of the medical professional completing the medical procedure has or has not exceeded the gaze duration threshold. As such, the CAD system can selectively modulate the bounding box 702C of the abnormality 314C to draw the attention of the medical professional completing the medical procedure thereto.

In some examples, the CAD system can have system parameters that prevent the bounding box 702 from modulating until some condition is met in addition to the gaze duration not meeting the gaze duration threshold. For example, if the operator is still intently focusing on the abnormality 314A or the abnormality 314B, the CAD system may not modulate the bounding box 702C around the abnormality 314C. Then, if the operator ceases to focus on the abnormality 314A or the abnormality 314B and begins scanning the screen, looking around the room, or moving the scope such that the abnormality 314C leaves the field of view being displayed, then the bounding box 702C around abnormality 314C can be updated.

In the example shown in FIG. 8, the CAD system can only display a bounding box 702 around or near a detected abnormality if the abnormality has been on the screen for a set amount of time, and the medical professional completing the medical procedure has not gazed at the detected anomoly over a threshold time (e.g., the gaze duration threshold 708). Such an implementation can mitigate negative operator experience that results from the bounding boxes 702 being placed around false positives. For example, some gastroenterologists have indicated that bounding boxes being placed over false positives (i.e., tissue areas being falsely identified by a CADe/CADx system as being of interest) causes distraction and annoyance during medical examinations.

For example, as shown in FIG. 8, a CAD system can identify or diagnose the abnormality 314A, the abnormality 314B, and the abnormality 314C, in the medical image 704. Display parameters of the CAD system can forego displaying any bounding boxes around (or other information in association with) individual ones of the tissue anomalies unless a gaze duration associated with such tissue anomalies does not meet a gaze duration threshold. For example, presume that the gaze of the operation duration on each of the abnormality 314A and the abnormality 314B exceeds the gaze duration threshold, whereas the operator does not gaze upon the abnormality 314C for at least the gaze duration threshold. As such, the CAD system does not place the bounding box 702A or the bounding box 702B around either of the abnormality 314A or the abnormality 314B, respectively, but does place the bounding box 702C around anomaly the abnormality 314C.

The gaze duration threshold 708 can be adjusted via the parameters of the system. For example, the gaze duration threshold 708 can be adjusted based on the type of medical procedure for which the systems can be used. The gaze duration threshold 708 can vary from 50 ms to 5 seconds. For example, the gaze duration threshold 708 can vary from 100 ms to 1 second. In yet another example, the gaze duration threshold 708 can be any amount of time that can be used to determine whether a doctor, or other medical professionals, looked at a detected abnormality, or if they potentially struggled to identify the detected abnormality.

In another example, the system can include one or more of the gaze duration threshold 708, each threshold of the one or more of the gaze duration threshold 708 can trigger the system to output or generate signals for different countermeasures. For example, if the medical professional looks at a detected anomaly over a gaze duration threshold, that anomaly can be labeled as complicated and flagged for review either by the medical professional completing the medical procedure or by a third-party medical professional. In another example, there can be a minimum the gaze duration threshold 708 that indicates different levels of review of the detected abnormality used. In another example, the gaze duration threshold 708 can be adjusted per the preferences of the medical professional conducting the medical procedure.

In yet another example, the gaze duration threshold (e.g., the gaze duration threshold 708) can change based on the identification of the CAD system. For example, the thresholds for an identified tissue abnormality can be determined based on classification data output from a CADx system. For example, a colon polyp that is analyzed by the CADx system and classified as being an inflammatory colon polyp can be assigned a first gaze duration threshold, whereas another colon polyp that is analyzed and classified as being a villous adenoma can be assigned a second gaze duration that is greater than the first gaze duration. Such examples can ensure that sufficient amounts of the operator's attention is given to tissue anomalies being more likely harmful to the patient.

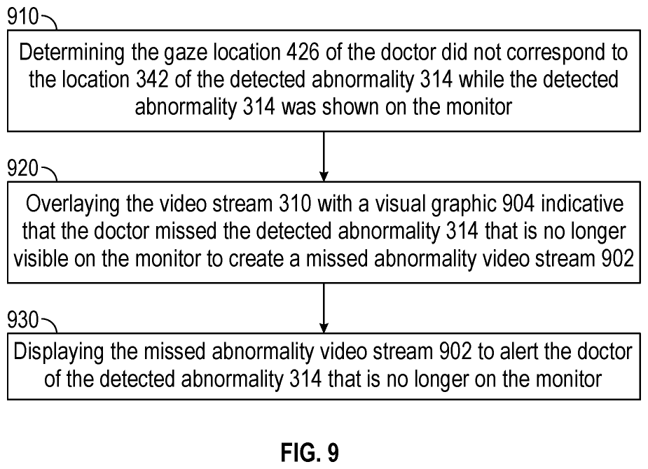

FIG. 9 is a flowchart further illustrating the method 500 from FIG. 5, according to an example of the present disclosure. For example, in addition to the generating the perceptible signal from step 610 from FIG. 6, the method 500 can also optionally include any of steps 910-930.

At step 910, step 610 of the method 500 can also include determining the gaze location of the doctor did not correspond to the location of the detected abnormality while the detected abnormality was shown on the monitor. For example, step 610 of the method 500 can also include determining the gaze location 426 of the doctor did not correspond to the location 342 of the detected abnormality 314 while the detected abnormality 314 was shown on the monitor (e.g., the display unit 18, the display 412, or the like). For example, the endoscopist can move the process camera such that the detected abnormality 314 is no longer shown on the display.

At step 920, step 610 of the method 500 can optionally include overlaying the video stream with a visual graphic indicative that the doctor missed the detected abnormality that is no longer visible on the monitor to create a missed abnormality video stream 902. For example, at step 920, step 610 of the method 500 can include overlaying the video stream 310 with a visual graphic 904. The visual graphic 904 can be an object, text, or any other indicator that can occur on the display to obtain the attention of the medical professional completing the medical procedure. In examples, the visual graphic 904 can be accompanied by a secondary alert, for example, audible, heptic, or any other alert that can accompany the visual graphic 904 to get the attention of the medical professional performing the medical procedure. The visual graphic 904 can be indicative that the doctor, or other medical professional completing the medical procedure, missed the detected abnormality 314, which is no longer visible on the monitor to create a missed abnormality video stream 902.

At step 930, step 610 of the method 500 can optionally include displaying the missed abnormality video stream to alert the doctor of the detected abnormality that is no longer on the monitor. For example, step 610 of the method 500 can optionally include displaying the missed abnormality video stream 902 to alert the doctor of the detected abnormality 314 that is no longer on the monitor. In examples, the controller 336 (FIG. 3), or the computer-aided diagnostic module 312 (FIG. 3) can transmit the missed abnormality video stream 902 to the user interface module 410 (FIG. 4), and the user interface module 410 can overlay the missed abnormality video stream 902 on the video stream 310. In another example, the controller 336, the computer-aided diagnostic module 312, or any other processor of, or in communication, with the systems can transmit the missed abnormality video stream 902 to any other component of, or in communication with, the system for storage, classification, storage, or to use as training data for future machine learning iterations.

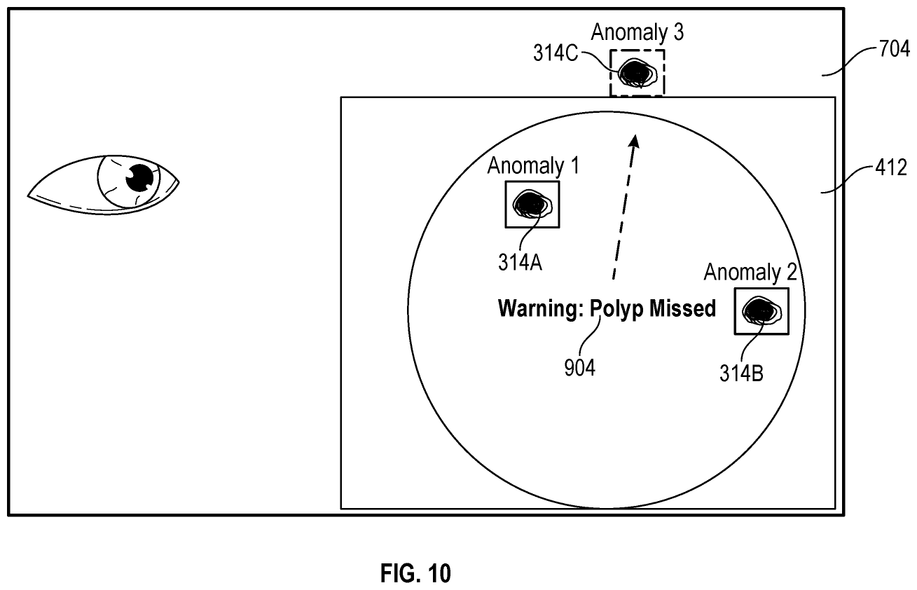

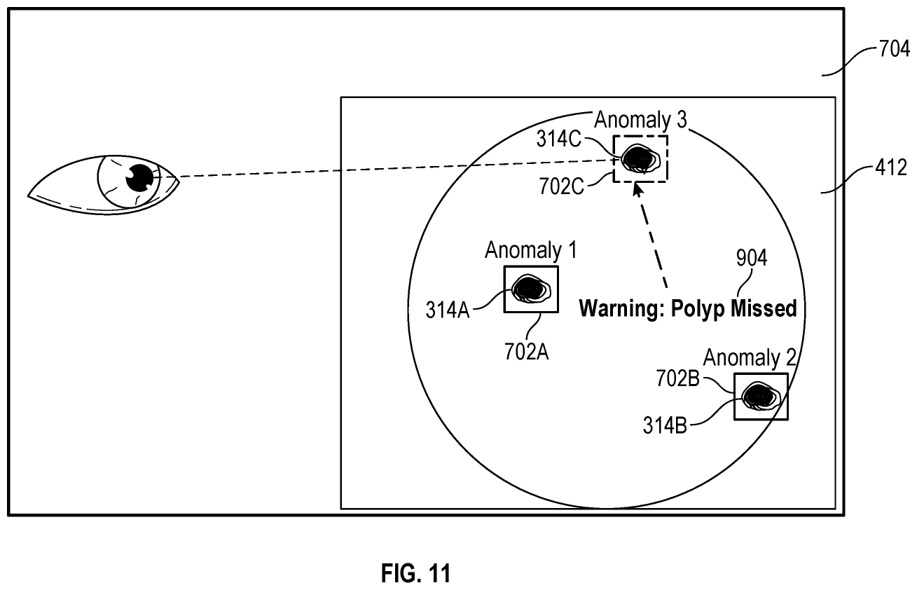

FIG. 10 is a schematic diagram illustrating an example display based on a gaze location of a medical professional, according to an example of the present disclosure. FIG. 11 is a schematic diagram illustrating an example display based on a gaze location of a medical professional, according to an example of the present disclosure. FIGS. 10 and 11 will be discussed concurrently below.

In an example implementation, audible or visible signals (e.g., the visual graphic 904) can direct the attention of the medical professional completing the medical procedure back to the detected abnormality 314, which have been displayed but not sufficiently examined before moving outside the displayed area (e.g., of the video stream 310 displayed on the display unit 18, or the display 412).

As shown in FIGS. 10 and 11, a CAD system (e.g., the system 300 or the system 400) can identify or diagnose the abnormality 314A, the abnormality 314B, and the abnormality 314C in a medical image 704. The operator may focus on each of abnormality 314A and abnormality 314B for at least their respective gaze duration thresholds (e.g., gaze duration threshold 708), but may fail to view abnormality 314C for its respective gaze duration threshold before adjusting the endoscope such that abnormality 314C is no longer displayed on a monitor or display (e.g., the display 412). In response to this determination, the computer-aided diagnostic module 312, the controller 336, or any other processor connected to the systems can generate a signal (e.g., the visual graphic 904) designed to draw the attention of the operator to abnormality 314C.

As shown on FIG. 10, the abnormality 314C can be out of displayed area, and the computer-aided diagnostic module 312, the controller 336, or any other processor in communication with the systems can generate the visual graphic 904 to draw attention of the endoscopist back in the direction that the detected abnormality 314 is off the screen. As shown in FIG. 10, the visual graphic 904 can include a warning label and a directional arrow. In another example, the warning label can include the unique identifier for the missed abnormality that can notify the medical professional of the unique identification, classification, and any other information that the detected abnormality 314 generates about the abnormality 314C. In examples, any other visual indicia can be used to alert the endoscopist of the missed abnormality.

As shown in FIG. 11, the abnormality 314C can be once again shown on the screen as the endoscopist moves the camera toward the abnormality 314C after the visual graphic 904 drew the endoscopists attention to the abnormality 314C. In such an implementation, the bounding box (e.g., the bounding box 702 (FIG. 7)) can turn from red to green once the gaze duration of the operator toward abnormality 314C reaches the appropriate gaze duration threshold (e.g., gaze duration threshold 708).

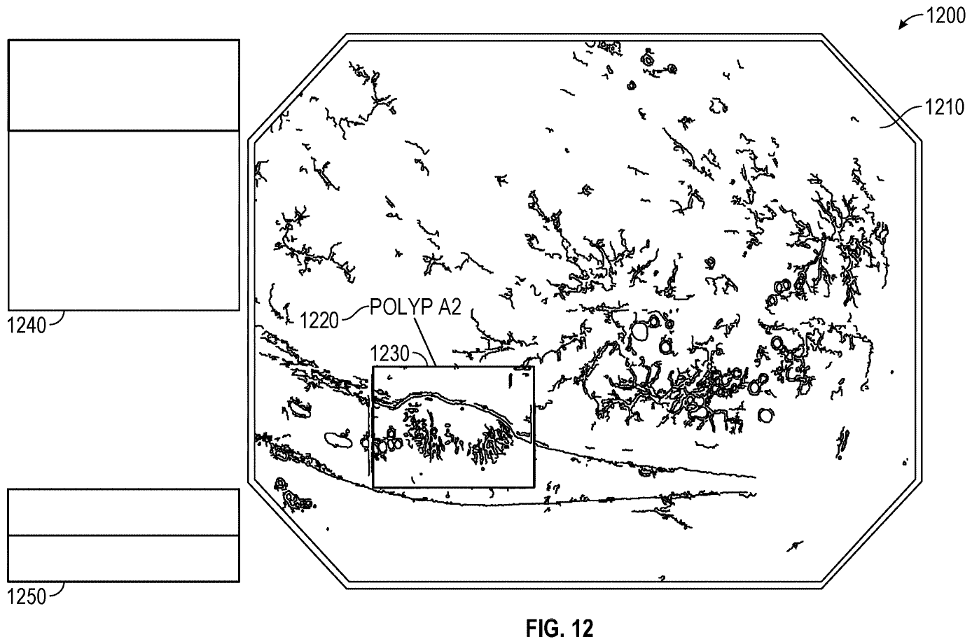

FIG. 12 illustrates a schematic diagram of an example of an annotated image 1200, which can optionally be displayed on a display within an operating room. The annotated image 1200 can for example be any of the annotated images discussed herein and can include an image 1210, an annotation 1220, a marking box 1230, a polyp identification box 1240, and a process identification box 1250. Moreover, the annotated image 1200 can be a symbolic representation of any video stream discussed herein. For example, as discussed above, any combination of the overlayed video stream 366, the analyzed video stream 414, the warning video stream 428, the overlaid video stream 430, the detected video stream 432, the detected and warning video stream 434, or the missed abnormality video stream 902 can be combined or overlayed on the video stream 310 by the user interface module 410 or any other controller or processor in communication with the system.

The image 1210 can be a continuous feed from the video stream captured by the camera during the endoscopic procedure. The image 1210 can be from a timestamp that corresponds a timestamp of an indicator of an abnormality found by the computer aided diagnostic system (e.g., the computer-aided diagnostic module 312 (FIG. 3). In another example, the system can automatically send a still example, or a collection of the image 1210 to the doctor for their review immediately following the medical procedure. For example, the image 1210 can be sent for review if the doctor did not look at the detected abnormality for a time greater than a threshold time, if the doctor looked at an abnormality for too long, and thus, indicates that the doctor could be confused or struggled to identify the abnormality, or for any other reason programmed by the system.

The annotation 1220 can be located on the image 1210, as shown in FIG. 12. In another example, the annotation 1220 can be off to the side of the image 1210, for example, in the polyp identification box 1240, the process identification box 1250, or any area around the image 1210. The annotation 1220 can be a unique identifier generated for the abnormality. The annotation 1220 can help identify a location of the abnormality encountered during the medical procedure.

The marking box 1230 (e.g., the bounding box 702 (FIG. 7)) can be overlayed the image 1210 to help identify the abnormality found. For example, the marking box 1230 can help a doctor during the procedure to find abnormalities that were found by the CAD system, or can help direct the doctor toward abnormalities that they may have missed during the medical procedure. In another example, the marking box 1230 can help the machine learning algorithm focus on the abnormality to improve the quality of learning.

The polyp identification box 1240 can include information about the abnormality from the medical procedure or from review by the doctor after the medical procedure. For example, the polyp identification box 1240 can include annotation of utterances made by the medical professional before and after the timestamp of the keyword being spoken. In another example, the polyp identification box 1240 can include notes typed in by the doctor after the doctor reviews the annotated image 1200. The information provided in the polyp identification box 1240 can help improve the machine learning by providing additional information about the annotated image 1200, which can help sort the annotated image 1200 into groupings of similar findings to improve the information being provided for the machine learning.

The process identification box 1250 can include process information about the medical procedure. For example, the process identification box 1250 can include a timestamp of the video stream that the image is captured from, a timestamp that the abnormality was recognized, a confidence level or the identification of the polyp, and any other processing information of the medical procedure that can be beneficial to know after the procedure is completed. The process identification box 1250 can also include manufacturing information or model numbers for the equipment used to perform the medical procedure.

The example of annotated image 1200 shown in FIG. 12 is just one example of the annotated image 1200. This example, including the information presented thereon is in no way intended to limit the scope of the invention. Rather, the provided information is intended to be a single example of the annotated image 1200 that the systems described herein can generate.

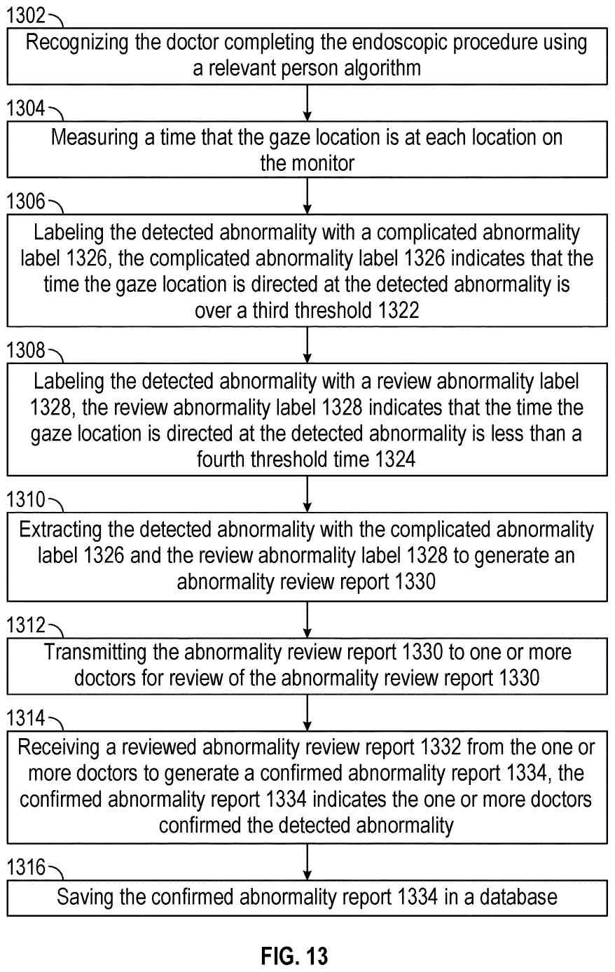

FIG. 13 is a flowchart that further describes the method 500 from FIG. 5, according to an example of the present disclosure. For example, the method 500 can optionally include steps 1302-1316 to generate a confirmed abnormality report 1320.

At step 1302, the method 500 can include recognizing the doctor completing the endoscopic procedure using a relevant person algorithm (e.g., the endoscopist detection algorithm 404 (FIG. 4). The relevant person algorithm can be configured to identify the doctor holding the endoscope to make sure the gaze location can be of the doctor performing the endoscopic procedure.

At step 1304, the method 500 can include measuring a time that the gaze location can be at each location on the monitor. As discussed above, the system can use an operator video stream, glasses, or any other sensor to determine a location of the doctors gaze, and can measure an amount of time the gaze of the doctor matches a location on the display at which the detected abnormality is shown. In examples, the system can determine that the medical professional has gazed at a location on the display for over a threshold value. If that location of the display does not have a pre-detected abnormality, or anoloy, that location of the screen can be flagged for future review. For example, this can indicate a false negative, for example, an abnormality that the medical professional found that the CAD system did not find in the video stream.

At step 1306, the method 500 can include labeling the detected abnormality with a complicated abnormality label, the complicated abnormality label indicates that the time the gaze location can be directed at the detected abnormality can be over a third threshold 1322. For example, if the doctor looks at the abnormality for a time over the third threshold 1322, the abnormality can be automatically labeled as a complicated abnormality 1326 because the increased amount of time of the gaze of the doctor being on the abnormality suggests that the doctor may have struggled to identify or classify the detected abnormality.

At step 1308, the method 500 can include labeling the detected abnormality with a review abnormality label 1328, the review abnormality label 1328 indicates that the time the gaze location can be directed at the detected abnormality can be less than a fourth threshold time 1324. For example, the fourth threshold time 1324 can be greater than the gaze duration threshold 708 but still a threshold number that raises concerns as to whether the doctor fully analyzed the abnormality.

At step 1310, the method 500 can include extracting the detected abnormality with the complicated abnormality 1326 label and the review abnormality label 1328 to generate an abnormality review report 1330.

At step 1312, the method 500 can include transmitting the abnormality review report 1330 to one or more doctors for review of the abnormality review report 1330. In examples, the abnormality review report 1330 can be sent to the doctor that completed the medical procedure, to one of their peers or supervisors, or to an unbiased third party for review.

At step 1314, the method 500 can include receiving a reviewed abnormality review report 1332 from the one or more doctors to generate a confirmed abnormality report 1334, the confirmed abnormality report 1334 indicates that one or more doctors confirmed the detected abnormality.

At step 1316, the method 500 can include saving the confirmed abnormality report 1334 in a database. In another example, the confirmed abnormality report 1334 can be transmitted to a convolutional neural network to train machine learning. In yet another example, the confirmed abnormality report 1334 can be transmitted to a patient's medical file that can stay with the patient as they age. In yet another example, the confirmed abnormality report 1334 can be stored, and can later be combined with pathology results of any abnormalities, or polyps, removed during the medical procedure.

In some examples, the gaze duration threshold (e.g., the gaze duration threshold 708, the third threshold 1322, or the fourth threshold time 1324) can change based on the identification of the CAD system. For example, the thresholds for an identified tissue abnormality can be determined based on classification data output from a CADx system. For example, a colon polyp that is analyzed by the CADx system and classified as being an inflammatory colon polyp can be assigned a first gaze duration threshold, whereas another colon polyp that is analyzed and classified as being a villous adenoma can be assigned a second gaze duration that is greater than the first gaze duration. Such examples can ensure that sufficient amounts of the operator's attention is given to tissue anomalies being more likely harmful to the patient.

In examples, images of the location that the doctor gazed over at threshold time, which did not have a corresponding detected anomaly or abnormality could also be saved. For example, these images can be saved and reviewed. If an abnormality or anomaly is confirmed, these images can be used to train the CAD system further to reduce false negatives on future medical procedures.

FIG. 14 is a block diagram illustrating an example of a machine upon which one or more examples can be implemented. Examples, as described herein, can include, or can operate by, logic or a number of components, or mechanisms in the machine 1400. Circuitry (e.g., processing circuitry) is a collection of circuits implemented in tangible entities of the machine 1400 that include hardware (e.g., simple circuits, gates, logic, etc.). Circuitry membership can be flexible over time. Circuitries include members that may, alone or in combination, perform specified operations when operating. In an example, hardware of the circuitry can be immutably designed to carry out a specific operation (e.g., hardwired). In an example, the hardware of the circuitry can include variably connected physical components (e.g., execution units, transistors, simple circuits, etc.) including a machine readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine readable medium elements are part of the circuitry or are communicatively coupled to the other components of the circuitry when the device is operating. In an example, any of the physical components can be used in more than one member of more than one circuitry. For example, under operation, execution units can be used in a first circuit of a first circuitry at one point in time and reused by a second circuit in the first circuitry, or by a third circuit in a second circuitry at a different time. Additional examples of these components with respect to the machine 1400 follow.

In alternative examples, the machine 1400 can operate as a standalone device or can be connected (e.g., networked) to other machines. In a networked deployment, the machine 1400 can operate in the capacity of a server machine, a client machine, or both in server-client network environments. In an example, the machine 1400 can act as a peer machine in peer-to-peer (P2P) (or other distributed) network environment. The machine 1400 can be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) 1400 can include a hardware processor 1402 (e.g., a central processing unit (CPU), a graphics processing unit (GPU), a hardware processor core, or any combination thereof), a main memory 1404, a static memory (e.g., memory or storage for firmware, microcode, a basic-input-output (BIOS), unified extensible firmware interface (UEFI), etc.) 1406, and mass storage 1408 (e.g., hard drives, tape drives, flash storage, or other block devices) some or all of which can communicate with each other via an interlink (e.g., bus) 1430. The machine 1400 can further include a display unit 1410, an alphanumeric input device 1412 (e.g., a keyboard), and a user interface (UI) navigation device 1414 (e.g., a mouse). In an example, the display unit 1410, input device 1412 and UI navigation device 1414 can be a touch screen display. The machine 1400 can additionally include a storage device (e.g., drive unit) 1408, a signal generation device 1418 (e.g., a speaker), a network interface device 1420, and one or more sensors 1416, such as a global positioning system (GPS) sensor, compass, accelerometer, or other sensor. The machine 1400 can include an output controller 1428, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 1402, the main memory 1404, the static memory 1406, or the mass storage 1408 can be, or include, a machine readable medium 1422 on which is stored one or more sets of data structures or instructions 1424 (e.g., software) embodying or utilized by any one or more of the techniques or functions described herein. The instructions 1424 can also reside, completely or at least partially, within any of registers of the processor 1402, the main memory 1404, the static memory 1406, or the mass storage 1408 during execution thereof by the machine 1400. In an example, one or any combination of the hardware processor 1402, the main memory 1404, the static memory 1406, or the mass storage 1408 can constitute the machine readable media 1422. While the machine readable medium 1422 is illustrated as a single medium, the term “machine readable medium” can include a single medium or multiple media (e.g., a centralized or distributed database, and/or associated caches and servers) configured to store the one or more instructions 1424.

The term “machine readable medium” can include any medium that is capable of storing, encoding, or carrying instructions for execution by the machine 1400 and that cause the machine 1400 to perform any one or more of the techniques of the present disclosure, or that is capable of storing, encoding or carrying data structures used by or associated with such instructions. Non-limiting machine readable medium examples can include solid-state memories, optical media, magnetic media, and signals (e.g., radio frequency signals, other photon based signals, sound signals, etc.). In an example, a non-transitory machine readable medium comprises a machine readable medium with a plurality of particles having invariant (e.g., rest) mass, and thus are compositions of matter. Accordingly, non-transitory machine-readable media are machine readable media that do not include transitory propagating signals. Specific examples of non-transitory machine readable media can include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

In an example, information stored or otherwise provided on the machine readable medium 1422 can be representative of the instructions 1424, such as instructions 1424 themselves or a format from which the instructions 1424 can be derived. This format from which the instructions 1424 can be derived can include source code, encoded instructions (e.g., in compressed or encrypted form), packaged instructions (e.g., split into multiple packages), or the like. The information representative of the instructions 1424 in the machine readable medium 1422 can be processed by processing circuitry into the instructions to implement any of the operations discussed herein. For example, deriving the instructions 1424 from the information (e.g., processing by the processing circuitry) can include: compiling (e.g., from source code, object code, etc.), interpreting, loading, organizing (e.g., dynamically or statically linking), encoding, decoding, encrypting, unencrypting, packaging, unpackaging, or otherwise manipulating the information into the instructions 1424.

In an example, the derivation of the instructions 1424 can include assembly, compilation, or interpretation of the information (e.g., by the processing circuitry) to create the instructions 1424 from some intermediate or preprocessed format provided by the machine readable medium 1422. The information, when provided in multiple parts, can be combined, unpacked, and modified to create the instructions 1424. For example, the information can be in multiple compressed source code packages (or object code, or binary executable code, etc.) on one or several remote servers. The source code packages can be encrypted when in transit over a network and decrypted, uncompressed, assembled (e.g., linked) if necessary, and compiled or interpreted (e.g., into a library, stand-alone executable etc.) at a local machine, and executed by the local machine.

The instructions 1424 can be further transmitted or received over a communications network 1426 using a transmission medium via the network interface device 1420 utilizing any one of a number of transfer protocols (e.g., frame relay, internet protocol (IP), transmission control protocol (TCP), user datagram protocol (UDP), hypertext transfer protocol (HTTP), etc.). Example communication networks can include a local area network (LAN), a wide area network (WAN), a packet data network (e.g., the Internet), LoRa/LoRaWAN, or satellite communication networks, mobile telephone networks (e.g., cellular networks such as those complying with 3G, 4G LTE/LTE-A, or 5G standards), Plain Old Telephone (POTS) networks, and wireless data networks (e.g., Institute of Electrical and Electronics Engineers (IEEE) 802.11 family of standards known as Wi-Fi®, IEEE 802.15.4 family of standards, peer-to-peer (P2P) networks, among others. In an example, the network interface device 1420 can include one or more physical jacks (e.g., Ethernet, coaxial, or phone jacks) or one or more antennas to connect to the communications network 1426. In an example, the network interface device 1420 can include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1400, and includes digital or analog communications signals or other intangible medium to facilitate communication of such software. A transmission medium is a machine-readable medium.

The following, non-limiting examples, detail certain aspects of the present subject matter to solve the challenges and provide the benefits discussed herein, among others.

Example 1 is a method for intelligent surveillance of attention and recognition of computer-aided diagnosis system outputs, the method comprising: receiving, with processing circuitry of a controller, a video stream from an endoscope including a camera, the video stream captured during an endoscopic procedure; receiving a signal from a computer-aided diagnostic module, the signal indicative of a detected abnormality within the video stream by a diagnostic algorithm; receiving an operator video stream from a camera installed within a room during the endoscopic procedure, the operator video stream includes, at least eyes of a doctor completing the endoscopic procedure; determining a gaze location of the doctor during the endoscopic procedure using a gaze algorithm, the gaze location indicative of a location on a monitor that the doctor looks at during the endoscopic procedure; determining whether the doctor looked at the detected abnormality by comparing the signal from the computer-aided diagnostic module and the gaze location of the doctor; and triggering a countermeasure based on determining that the doctor did not look at the detected abnormality.

In Example 2, the subject matter of Example 1 includes, recognizing the doctor completing the endoscopic procedure using a relevant person algorithm, wherein the relevant person algorithm is configured to identify the doctor holding the endoscope to make sure the gaze location is of the doctor performing the endoscopic procedure.

In Example 3, the subject matter of Example 2 includes, measuring a time that the gaze location is at each location on the monitor; labeling the detected abnormality with a complicated abnormality label, the complicated abnormality label indicates that the time the gaze location is directed at the detected abnormality is over a third threshold; labeling the detected abnormality with a review abnormality label, the review abnormality label indicates that the time the gaze location is directed at the detected abnormality is less than a fourth threshold time; and extracting the detected abnormality with the complicated abnormality label and the review abnormality label to generate an abnormality review report.

In Example 4, the subject matter of Example 3 includes, transmitting the abnormality review report to one or more doctors for review of the abnormality review report; receiving the reviewed abnormality review report from the one or more doctors to generate a confirmed abnormality report, the confirmed abnormality report indicates the one or more doctors confirmed the detected abnormality; and saving the confirmed abnormality report in a database.

In Example 5, the subject matter of Examples 1-4 includes, wherein triggering the countermeasure includes transmitting a warning signal.

In Example 6, the subject matter of Example 5 includes, wherein the warning signal indicates that the gaze location of the doctor was not directed at the detected abnormality for a first set threshold time.

In Example 7, the subject matter of Examples 5-6 includes, wherein the warning signal includes an abnormality identification unique to each detected abnormality by the computer-aided diagnostic module.

In Example 8, the subject matter of Examples 1-7 includes, wherein triggering the countermeasure comprises: generating a perceptible signal, the perceptible signal configured to notify the doctor of the detected abnormality.

In Example 9, the subject matter of Example 8 includes, wherein the perceptible signal includes the processing circuitry of the controller processing the video stream with the detected abnormality by: overlaying the video stream with a bounding box by corresponding a location of the detected abnormality from the computer-aided diagnostic module and a location of the detected abnormality on the video stream to create an overlayed video stream; and displaying the overlayed video stream to indicate to the doctor the location of the detected abnormality.

In Example 10, the subject matter of Example 9 includes, wherein the bounding box is a first color before the gaze location of the doctor is directed at the detected abnormality for a set threshold time, and wherein the bounding box is a second color after the gaze location of the doctor is directed at the detected abnormality for the set threshold time.

In Example 11, the subject matter of Examples 8-10 includes, wherein the perceptible signal includes a bounding box surrounding the detected abnormality when the gaze location of the doctor does not align with a location of the detected abnormality.

In Example 12, the subject matter of Example 11 includes, wherein the perceptible signal includes the processing circuitry of the controller processing the video stream with the detected abnormality by: determining the gaze location of the doctor did not correspond to the location of the detected abnormality while the detected abnormality was shown on the monitor; overlaying the video stream with a visual graphic indicative that the doctor missed the detected abnormality that is no longer visible on the monitor to create a missed abnormality video stream; and displaying the missed abnormality video stream to alert the doctor of the detected abnormality that is no longer on the monitor.

Example 13 is a system for intelligent surveillance of attention and recognition of computer-aided diagnosis system outputs, the system comprising: an endoscope comprising: an elongated member including a distal portion, the elongated member comprising: a process camera attached to the distal portion, the process camera capturing a video stream during a procedure; a computer-aided diagnostic module configured to detect an abnormality within the video stream using a diagnostic algorithm and transmit a signal; a camera to capture an operator video stream, the operator video stream including at least eyes of a doctor during the procedure; a memory including instructions; and a controller including processing circuitry that, when in operation, is configured by the instructions to: determine a gaze location of the doctor during the procedure using a gaze algorithm, the gaze location indicative of a location on a monitor that the doctor looks at during the procedure; determine whether the doctor looked at the detected abnormality by comparing the signal from the computer-aided diagnostic module and the gaze location of the doctor; and trigger a countermeasure based on determining that the doctor did not look at the detected abnormality.

In Example 14, the subject matter of Example 13 includes, recognizing the doctor completing the procedure using a relevant person algorithm, wherein the relevant person algorithm is configured to identify the doctor holding the endoscope to make sure the gaze location is of the doctor performing the procedure.

In Example 15, the subject matter of Example 14 includes, wherein the processing circuitry of the controller is configured by the instructions to: measure a time that the gaze location is at each location on the monitor; label the detected abnormality with a complicated abnormality label, the complicated abnormality label indicates that the time the gaze location is directed at the detected abnormality is over a third threshold; label the detected abnormality with a review abnormality label, the review abnormality label indicates that the time the gaze location is directed at the detected abnormality is less than a fourth threshold time; and extract the detected abnormality with the complicated abnormality label and the review abnormality label to generate an abnormality review report.

In Example 16, the subject matter of Example 15 includes, wherein the processing circuitry of the controller is configured by the instructions to: transmit the abnormality review report to one or more doctors for review of the abnormality review report; receive the reviewed abnormality review report from the one or more doctors to generate a confirmed abnormality report, the confirmed abnormality report indicates the one or more doctors confirmed the detected abnormality; and save the confirmed abnormality report in a database.

In Example 17, the subject matter of Examples 13-16 includes, wherein triggering the countermeasure includes transmitting a warning signal, and wherein the warning signal indicates that the gaze location of the doctor was not directed at the detected abnormality for a first set threshold time.

In Example 18, the subject matter of Example 17 includes, wherein the warning signal includes an abnormality identification unique to each detected abnormality by the computer-aided diagnostic module.

In Example 19, the subject matter of Examples 13-18 includes, wherein to trigger the countermeasure, the processing circuitry of the controller is configured by the instructions to: generate a perceptible signal, the perceptible signal configured to notify the doctor of the detected abnormality.

In Example 20, the subject matter of Example 19 includes, wherein to generate the perceptible signal the processing circuitry of the controller is configured by the instructions to: overlay the video stream with a bounding box by corresponding to a location of the detected abnormality from the computer-aided diagnostic module and a location of the detected abnormality on the video stream to create an overlayed video stream; and display the overlayed video stream to indicate to the doctor the location of the detected abnormality.

In Example 21, the subject matter of Example 20 includes, wherein the bounding box is a first color before the gaze location of the doctor is directed at the detected abnormality for a set threshold time, and wherein the bounding box is a second color after the gaze location of the doctor is directed at the detected abnormality for the set threshold time.

In Example 22, the subject matter of Examples 19-21 includes, wherein the perceptible signal includes a bounding box surrounding the detected abnormality when the gaze location of the doctor does not align with a location of the detected abnormality.

In Example 23, the subject matter of Example 22 includes, wherein to generate the perceptible signal the processing circuitry of the controller is configured by the instructions to: determine the gaze location of the doctor did not correspond to the location of the detected abnormality while the detected abnormality was shown on the monitor; overlay the video stream with a visual graphic indicative that the doctor missed the detected abnormality that is no longer visible on the monitor to create a missed abnormality video stream; and display the missed abnormality video stream to alert the doctor of the detected abnormality that is no longer on the monitor.

Example 24 is at least one machine-readable medium including instructions that, when executed by processing circuitry, cause the processing circuitry to perform operations to implement of any of Examples 1-23.

Example 25 is an apparatus comprising means to implement of any of Examples 1-23.

Example 26 is a system to implement of any of Examples 1-23.

Example 27 is a method to implement of any of Examples 1-23.

The above-detailed description includes references to the accompanying drawings, which form a part of the detailed description. The drawings show, by way of illustration, specific examples that can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The above description is intended to be illustrative, and not restrictive. For example, the above-described examples (or one or more aspects thereof) can be used in combination with each other. Other examples can be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed example. Thus, the following claims are hereby incorporated into the Detailed Description, with each claim standing on its own as a separate example. The scope of the examples should be determined with reference to the appended claims, along with the full scope of equivalents to which such claims are entitled.

1. A method for intelligent surveillance of attention and recognition of computer-aided diagnosis system outputs, the method comprising:
receiving, with processing circuitry of a controller, a video stream from an endoscope including a camera, the video stream captured during an endoscopic procedure;
receiving a signal from a computer-aided diagnostic module, the signal indicative of a detected abnormality within the video stream by a diagnostic algorithm;
receiving an operator video stream from a camera installed within a room during the endoscopic procedure, the operator video stream includes at least eyes of a doctor completing the endoscopic procedure;
determining a gaze location of the doctor during the endoscopic procedure using a gaze algorithm, the gaze location indicative of a location on a monitor that the doctor looks at during the endoscopic procedure;
determining whether the doctor looked at the detected abnormality by comparing the signal from the computer-aided diagnostic module and the gaze location of the doctor; and
triggering a countermeasure based on determining that the doctor did not look at the detected abnormality.

2. The method of claim 1, comprising:
recognizing the doctor completing the endoscopic procedure using a relevant person algorithm, wherein the relevant person algorithm is configured to identify the doctor holding the endoscope to make sure the gaze location is of the doctor performing the endoscopic procedure.

3. The method of claim 2, comprising:
measuring a time that the gaze location is at each location on the monitor;
labeling the detected abnormality with a complicated abnormality label, the complicated abnormality label indicates that the time the gaze location is directed at the detected abnormality is over a third threshold;
labeling the detected abnormality with a review abnormality label, the review abnormality label indicates that the time the gaze location is directed at the detected abnormality is less than a fourth threshold time; and
extracting the detected abnormality with the complicated abnormality label and the review abnormality label to generate an abnormality review report.

4. The method of claim 3, comprising:
transmitting the abnormality review report to one or more doctors for review of the abnormality review report;
receiving the reviewed abnormality review report from the one or more doctors to generate a confirmed abnormality report, the confirmed abnormality report indicates the one or more doctors confirmed the detected abnormality; and
saving the confirmed abnormality report in a database.

5. The method of claim 1, wherein triggering the countermeasure includes transmitting a warning signal.

6. The method of claim 5, wherein the warning signal indicates that the gaze location of the doctor was not directed at the detected abnormality for a first set threshold time.

7. The method of claim 5, wherein the warning signal includes an abnormality identification unique to each detected abnormality by the computer-aided diagnostic module.

8. The method of claim 1, wherein triggering the countermeasure comprises:
generating a perceptible signal, the perceptible signal configured to notify the doctor of the detected abnormality.

9. The method of claim 8, wherein the perceptible signal includes the processing circuitry of the controller processing the video stream with the detected abnormality by:
overlaying the video stream with a bounding box by corresponding a location of the detected abnormality from the computer-aided diagnostic module and a location of the detected abnormality on the video stream to create an overlayed video stream; and
displaying the overlayed video stream to indicate to the doctor the location of the detected abnormality.

10. The method of claim 9, wherein the bounding box is a first color before the gaze location of the doctor is directed at the detected abnormality for a set threshold time, and wherein the bounding box is a second color after the gaze location of the doctor is directed at the detected abnormality for the set threshold time.

11. The method of claim 8, wherein the perceptible signal includes a bounding box surrounding the detected abnormality when the gaze location of the doctor does not align with a location of the detected abnormality.

12. The method of claim 11, wherein the perceptible signal includes the processing circuitry of the controller processing the video stream with the detected abnormality by:
determining the gaze location of the doctor did not correspond to the location of the detected abnormality while the detected abnormality was shown on the monitor;
overlaying the video stream with a visual graphic indicative that the doctor missed the detected abnormality that is no longer visible on the monitor to create a missed abnormality video stream; and
displaying the missed abnormality video stream to alert the doctor of the detected abnormality that is no longer on the monitor.

13. A system for intelligent surveillance of attention and recognition of computer-aided diagnosis system outputs, the system comprising:
an endoscope comprising:
an elongated member including a distal portion, the elongated member comprising:
a process camera attached to the distal portion, the process camera capturing a video stream during a procedure;
a computer-aided diagnostic module configured to detect an abnormality within the video stream using a diagnostic algorithm and transmit a signal;
a camera to capture an operator video stream, the operator video stream including at least eyes of a doctor during the procedure;
a memory including instructions; and
a controller including processing circuitry that, when in operation, is configured by the instructions to:
determine a gaze location of the doctor during the procedure using a gaze algorithm, the gaze location indicative of a location on a monitor that the doctor looks at during the procedure;
determine whether the doctor looked at the detected abnormality by comparing the signal from the computer-aided diagnostic module and the gaze location of the doctor; and
trigger a countermeasure based on determining that the doctor did not look at the detected abnormality.

14. The system of claim 13, comprising:
recognizing the doctor completing the procedure using a relevant person algorithm, wherein the relevant person algorithm is configured to identify the doctor holding the endoscope to make sure the gaze location is of the doctor performing the procedure.

15. The system of claim 14, wherein the processing circuitry of the controller is configured by the instructions to:
measure a time that the gaze location is at each location on the monitor;
label the detected abnormality with a complicated abnormality label, the complicated abnormality label indicates that the time the gaze location is directed at the detected abnormality is over a third threshold;
label the detected abnormality with a review abnormality label, the review abnormality label indicates that the time the gaze location is directed at the detected abnormality is less than a fourth threshold time; and
extract the detected abnormality with the complicated abnormality label and the review abnormality label to generate an abnormality review report.

16. The system of claim 15, wherein the processing circuitry of the controller is configured by the instructions to:
transmit the abnormality review report to one or more doctors for review of the abnormality review report;
receive the reviewed abnormality review report from the one or more doctors to generate a confirmed abnormality report, the confirmed abnormality report indicates the one or more doctors confirmed the detected abnormality; and
save the confirmed abnormality report in a database.

17. The system of claim 13, wherein triggering the countermeasure includes transmitting a warning signal, and wherein the warning signal indicates that the gaze location of the doctor was not directed at the detected abnormality for a first set threshold time.

18. The system of claim 17, wherein the warning signal includes an abnormality identification unique to each detected abnormality by the computer-aided diagnostic module.

19. The system of claim 13, wherein to trigger the countermeasure, the processing circuitry of the controller is configured by the instructions to:
generate a perceptible signal, the perceptible signal configured to notify the doctor of the detected abnormality.

20. The system of claim 19, wherein to generate the perceptible signal the processing circuitry of the controller is configured by the instructions to:
overlay the video stream with a bounding box by corresponding to a location of the detected abnormality from the computer-aided diagnostic module and a location of the detected abnormality on the video stream to create an overlayed video stream; and
display the overlayed video stream to indicate to the doctor the location of the detected abnormality.

21. The system of claim 20, wherein the bounding box is a first color before the gaze location of the doctor is directed at the detected abnormality for a set threshold time, and wherein the bounding box is a second color after the gaze location of the doctor is directed at the detected abnormality for the set threshold time.

22. The system of claim 19, wherein the perceptible signal includes a bounding box surrounding the detected abnormality when the gaze location of the doctor does not align with a location of the detected abnormality.

23. The system of claim 22, wherein to generate the perceptible signal the processing circuitry of the controller is configured by the instructions to:
determine the gaze location of the doctor did not correspond to the location of the detected abnormality while the detected abnormality was shown on the monitor;
overlay the video stream with a visual graphic indicative that the doctor missed the detected abnormality that is no longer visible on the monitor to create a missed abnormality video stream; and
display the missed abnormality video stream to alert the doctor of the detected abnormality that is no longer on the monitor.
